(19) United States
(12) Patent Application Publication
(10) Pub. No.: US 2006/0035360 A1
(43) Pub. Date: Feb. 16, 2006

(54) Thermostable DNA polymerases and methods of making same

(75) Inventor: Joseph W. Farchaus III, Bloomsbury, NJ (US)

Correspondence Address: AMERSHAM BIOSCIENCES;PATENT DEPARTMENT, 800 CENTENNIAL AVENUE, PISCATAWAY, NJ, 08855, US

(73) Assignee: Amersham Biosciences Corp

(21) Appl. No.: 11/242,730

(22) Filed: Oct. 4, 2005

Related U.S. Application Data
(62) Division of application No. 10/126,757, filed on Apr. 19, 2002.
(60) Provisional application No. 60/340,733, filed on Oct. 30, 2001.

(51) Int. Cl. C12N 9/22; C12P 21/06; C12N 15/74; C12N 1/21

(52) U.S. Cl. 435/199; 435/183; 435/69.1; 435/320.1; 435/252.3

(57) ABSTRACT

The present invention relates to methods and compositions for providing purified thermostable enzymes, particularly thermostable DNA polymerases, that are free of exogenous detergents. The present invention also provides methods for providing such purified thermostable DNA polymerases to assays in an active form by adding one or more detergents. The present invention further provides compositions and kits comprising purified thermostable DNA polymerases for use in a variety of applications, including amplification and sequencing of nucleic acids.

### CROSS-REFERENCE TO RELATED PATENT APPLICATION

This application is a divisional application of U.S. patent application Ser. No. 10/126,757 filed Apr. 19, 2002, and claims priority to U.S. provisional patent application No. 60/340,733, filed Oct. 30, 2001, the disclosures of which are hereby incorporated by reference in their entireties.

### BACKGROUND OF THE INVENTION

The present invention relates to thermostable DNA polymerases, compositions and kits comprising thermostable DNA polymerases, and methods for isolating and using thermostable DNA polymerases.

DNA polymerases are enzymes that catalyze the template-directed synthesis of DNA from deoxyribonucleoside triphosphates. Typically, DNA polymerases (e.g., DNA polymerases I, II, and III in microorganisms; DNA polymerases α, β, and γ, in animal cells) direct the synthesis of a DNA strand from a DNA template; however, some DNA polymerases (referred to generally as “reverse transcriptases”) direct the synthesis of a DNA strand from an RNA template. Generally, these are recognized by the IUPAC-IUBMB Joint Commission on Biochemical Nomenclature under the Enzyme Commission numbers EC 2.7.7.7 and EC 2.7.7.49. Extensive research has been conducted on isolation and characterization of DNA polymerases from various organisms, including bacteria, yeast, and humans, particularly for use in in vitro reactions.

When selecting a DNA polymerase for use in a particular in vitro reaction, the skilled artisan must consider a number of variables. For example, a DNA polymerase may be selected to have its natural 5′-3′ or 3′-5′ exonuclease activity deleted (e.g., by mutagenesis or by post-translational modification such as enzymatic digestion), to exhibit a low error rate, to exhibit high processivity and elongation rate, and/or to exhibit advantageous thermal stability. The identification of DNA polymerases from thermophilic microorganisms, and the use of thermostable DNA polymerases in methods such as PCR, have led to a revolution in the ability to identify and manipulate DNA. A number of thermostable DNA polymerases have been isolated from thermophilic eubacteria, thermophilic archaea, and others.

Examples of thermostable DNA polymerases include but not limited to Taq DNA polymerase derived from Thermus aquaticus (see, e.g., U.S. Pat. No. 4,889,818); Tth DNA polymerase derived from Thermus thermophilus (see, e.g., U.S. Pat. Nos. 5,192,674; 5,242,818; 5,413,926); Tsp sps17 DNA polymerase derived from Thermus species sps 17, now called Thermus oshimai (see, e.g., U.S. Pat. No. 5,405,774); Pfu DNA polymerase derived from Pyrococcus furiosus (U.S. Pat. No. 5,948,663); Bst DNA polymerase derived from Bacillus stearothermophilus (U.S. Pat. No. 5,747,298); Tli DNA polymerase derived from Thermococcus litoralis (U.S. Pat. No. 5,322,785); KOD DNA polymerase derived from Pyrococcus sp. KOD1 (U.S. Pat. No. 6,033,859); nTha and Tha DNA polymerase derived from Thermococcus barosii (U.S. Pat. Nos. 5,602,011 and 5,882,904); and commercially available DNA polymerases such as Thermo Sequenase (Amersham) and AmpliTaq (Applied Biosystems, Tabor, S. & Richardson, C. C. (1995) Proc. Natl. Acad. Sci. USA 92, 6339-6343).

Detergents are widely used in the art to solubilize membranes, to enhance permeabilization effects of various chemical agents, and for disruption of the bacterial cell walls, facilitating the preparation of intracellular proteins, such as DNA polymerases, from microorganisms. Goldstein et. al. discloses methods of making a thermostable enzyme which is substantially free of nucleic acids (U.S. Pat. No. 5,861,295). Gelfand et al. discloses a stable enzyme composition comprising a purified, stable thermostable polymerase in a buffer containing one or more non-ionic polymeric detergents (U.S. Pat. No. 6,127,155). Simpson et al., Biochem. Cell Biol. 68: 1292-6 (1990) discloses purification of a DNA polymerase that is stabilized by additives such as Triton X-100.

Detergents can be difficult to remove completely from the resulting purified species. Additionally, in enzymatic reactions, such as DNA sequencing reactions, the presence of detergents may affect results. See, e.g., Ruiz-Martinez et al., Anal. Chem. 70: 1516-1527, 1998. Additionally, some thermostable DNA polymerases may substantially decrease in activity over time in the absence of detergents. See, e.g., U.S. Pat. No. 6,127,155.

### SUMMARY OF THE INVENTION

The present invention relates to compositions and methods that permit the skilled artisan to control the environment in which thermostable enzymes, in particular thermostable DNA polymerases, are purified and used. In a first aspect, the present invention provides methods for purifying thermostable enzymes without the addition of an exogenous detergent. In a related aspect, the present invention provides compositions comprising a purified thermostable enzyme free from exogenously added detergents.

Preferably, a thermostable enzyme is a thermostable DNA polymerase, and is most preferably obtained or derived from a microorganism of a genus selected from the group consisting of Thermus, Pyrococcus, Thermococcus, Aquifex, Sulfolobus, Thermoplasma, Thermoanaerobacter, Rhodothermus, Methanococcus, and Thermotoga.

The thermostable enzymes of the present invention can be obtained from any source and can be a native or recombinant protein. Thus, the phrase “derived from” as used in this paragraph is intended to indicate that the thermostable DNA polymerase is expressed recombinantly, and the expressed DNA sequence is a wild-type sequence obtained from a thermophilic organism, or a mutated form thereof. Examples of suitable organisms providing a source of thermostable DNA polymerase (sequences and/or proteins) include Thermus flavus, Thermus ruber, Thermus thermophilus, Bacillus stearothermophilus, Thermus aquaticus, Thermus lacteus, Meiothermus ruber, Thermus oshimai, Methanothermusfervidus, Sulfolobus solfataricus, Sulfolobus acidocaldarius, Thermoplasma acidophilum, Methanobacterium thermoautotrophicum and Desulfurococcus mobilis.

Preferred DNA polymerases include, but are not limited to, Taq DNA polymerase; Tth DNA polymerase; Pfu DNA polymerase; Bst DNA polymerase; Tli DNA polymerase; KOD DNA polymerase; nTha and/or Tba DNA polymerase. In certain embodiments, the thermostable DNA polymerases of the present invention have been modified by deletion, substitution, or addition of one or more amino acids in comparisaon to a wild-type sequence, such as Taq Δ271 F667Y, Tth Δ273 F668Y, and Taq Δ271 F667Y E681W. Particularly preferred DNA polymerases are provided hereinafter in Table 1.

Thermostable DNA polymerases are preferably purified from cells that either naturally express the enzyme, or that have been engineered to express the enzyme (e.g., an E. coli expressing an exogenous DNA polymerase such as Taq DNA polymerase). These methods comprise lysing the cells in an environment into which exogenous detergent has not been added, and then purifying the DNA polymerase by one or more purification steps, again in the absence of exogenously added detergent. A substantially purified DNA polymerase obtained from such a method is free from any exogenous detergent.

In various preferred embodiments, the purification methods of the present invention comprise one or more of the following steps: (i) heating a cell lysate to denature one or more proteins; (ii) centrifuging the cell lysate to remove all or a portion of the supernatant to provide a clarified lysate; and (iii) fractionating the clarified lysate using a chromatography medium, most preferably a chromatography medium comprising a butyl functionality.

The term “thermostable” refers to an enzyme that retains activity at a temperature greater than 50° C.; thus, a thermostable DNA polymerase retains the ability to direct synthesis of a DNA strand at this elevated temperature. An enzyme may have more than one enzymatic activity. For example, a DNA polymerase may also comprise endonuclease and/or exonuclease activities. Such an enzyme may exhibit thermostability with regard to one activity, but not another.

Preferably, a thermostable enzyme retains activity at a temperature between about 50° C. and 80° C., more preferably between about 55° C. and 75° C.; and most preferably between about 60° C. and 70° C. In addition, the activity exhibited at one of these elevated temperatures is preferably greater than the activity of the same enzyme at 37° C. in the same environmental milieu (e.g., in the same buffer composition). Thus, particularly preferred thermostable enzymes exhibit maximal catalytic activity at a temperature between about 60° C. and 95° C., most preferably at a temperature between about 70° C. and 80° C. The term “about” in this context refers to +/− 10% of a given temperature.

The term “active” as used herein refers to the ability of an enzyme to catalyze a chemical reaction. An enzyme will have a maximal activity rate, which is preferably measured under conditions of saturating substrate concentration and at a selected set of environmental conditions including temperature, pH and salt concentration. For the DNA polymerases described herein, preferred conditions for measuring activity are 25 mM TAPS (tris-hydroxymethyl-methylaminopropane sulfonic acid) buffer, pH 9.3 (measured at 25° C.), 50 mM KCl, 2 mM MgCl₂, 1 mM 2-mercaptoethanol, 0.2 mM each of dGTP, dCTP, dTTP, 0.2 mM [α-³³P]-dATP (0.05-0.1 Ci/mmol) and 0.4 mg/mL activated salmon sperm DNA. The reaction is allowed to proceed at 74° C. Exemplary methods for measuring the DNA polymerase activity of an enzyme under such conditions are provided hereinafter.

The term “inactive” as used herein refers to an activity that is less than 10%, more preferably less than 5%, and most preferably less than 1% of the maximal activity rate for the enzyme. For the DNA polymerases described herein, this preferably refers to comparing an activity to the rate obtained under the preferred conditions for measuring activity described in the preceding paragraph.

Most preferably, the thermostable enzymes of the present invention are not irreversibly inactivated when subjected to the purification steps required to obtain compositions comprising a purified thermostable enzyme free from exogenously added detergents. “Irreversible inactivation” for purposes herein refers to a loss of enzymatic activity that cannot be recovered by altering the conditions to which the enzyme is exposed. Thus, a composition may comprise an inactive themostable enzyme, so long as the enzyme can be activated subsequently by altering its environment (e.g., by subsequent exposure to detergent, by an increase in temperature, etc.).

Themostable DNA polymerases preferably are not irreversibly inactivated under conditions required for use in DNA amplification methods, such as PCR. During PCR, for example, a polymerase may be subjected to repeated cycles of heating and cooling required for melting and annealing complementary DNA strands. Such conditions may depend, e.g., on the buffer salt concentration and composition and the length and nucleotide composition of the nucleic acids being amplified or used as primers, but typically the highest temperature used ranges from about 90° C. to about 105° C. for typically about 0.5 to four minutes. Increased temperatures may be required as the buffer salt concentration and/or GC composition of the nucleic acid is increased. Preferably, the enzyme does not become irreversible denatured at temperatures up to 90° C., more preferably up to 95° C., even more preferably up to 98° C., and most preferably up to 100° C. The ability to withstand increased temperature is also often expressed in terms of a “half-life,” referring to the time at a given temperature when the enzymatic activity of a given amount of enzyme has been reduced to half of the original activity. Preferably, the enzyme has a half-life of greater than 30 minutes at 90° C.,

The term “detergent” as used herein refers to amphipathic surface-active agents (“surfactants”) that, when added to a liquid, reduce surface tension of the liquid in comparison to the same liquid in the absence of the detergent. See, e.g., Detergents: A guide to the properties and uses of detergents in biological systems, Calbiochem-Novabiochem Corporation, 2001, which is hereby incorporated by reference in its entirety.

The skilled artisan will understand that various components that are naturally present in organisms may exhibit detergent-like behavior. Thus, the term “exogenously added detergent” refers to a detergent that is not endogenously present in an organism being processed in a particular method. Detergents are commonly added from an exogenous source for solubilization of membrane proteins and for facilitating chemical disruption of cells in order to extract intracellular proteins.

Typical detergents used for this purpose include, but are not limited to, anionic detergents such as sodium n-dodecyl sulfate (SDS); and dihydroxy or trihydroxy bile acids (and their salts), such as cholic acid (sodium cholate), deoxycholic acid (sodium deoxycholate), taurodeoxycholic acid (sodium taurodeoxycholate), taurocholic acid (sodium taurocholate), glycodeoxycholic acid (sodium glycodeoxycholate), glycocholic acid (sodium glycocholate); cationic detergents such as cetyl trimethyl-ammonium bromide (CTAB); non-ionic detergents such as the polyoxyethylenes NP-40, TRITON® X-100, TRITON® X114, C₁₂E₈, C₁₂E₉, GENAPOL® X-080, GENAPOL® X-100, LUBROL® PX, BRIJ® 35, TWEEN® 20, and TWEEN® 20; alkyl glycosides such as dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, and n-hexyl-β-D-glucopyranoside; alkylamine oxides such as lauryl dimethylamine oxide (LDAO); and zwitterionic detergents, such as CHAPS, CHAPSO, n-dodecyl-N,N-dimethylglycine, and ZWITTERGENTS® 3-08, 3-10, 3-12, 3-14, and 3-16. The present invention relates to purified and substantially purified compositions that are free of any of these exemplary detergents.

The term “purified” as used herein with reference to enzymes does not refer to absolute purity. Rather, “purified” is intended to refer to a substance in a composition that contains fewer protein species other than the enzyme of interest in comparison to the organism from which it originated. Preferably, an enzyme is “substantially pure,” indicating that the enzyme represents at least 50% of protein on a mass basis of the composition comprising the enzyme. More preferably, a substantially pure enzyme is at least 75% on a mass basis of the composition, and most preferably at least 95% on a mass basis of the composition.

In another aspect, the present invention provides methods for providing a purified thermostable DNA polymerase to an assay. These methods comprise adding one or more detergents to a composition comprising a purified thermostable DNA polymerase, where the composition comprising the purified thermostable DNA polymerase was previously free of exogenously added detergent. Most preferably, adding detergent to a purified thermostable DNA polymerase that was previously free of exogenously added detergent converts an inactive DNA polymerase to an active form, or increases the activity of a DNA polymerase.

In various aspects, one or more detergents may be added to the compositions described above, and the resulting composition may be added to a reaction mixture for use in an assay; alternatively, a purified thermostable DNA polymerase may be added to a reaction mixture and the detergent may be added subsequently; and/or detergent may be added to a reaction mixture and the thermostable DNA polymerase may be added subsequently. In any case, the result is that a purified thermostable DNA polymerase that was previously free of exogenously added detergent is now in a composition comprising detergent.

The term “assay” as used herein refers to any reaction mixture in which a purified thermostable DNA polymerase catalyzes the template-directed synthesis of DNA from deoxyribonucleotide triphosphates or analogues such as dideoxyribonucleotide triphosphates. Preferred assays include DNA polymerase activity assays, single- or double-stranded exonuclease activity assays, single- or double-stranded endonuclease activity assays, nucleic acid amplification reactions, and nucleic acid sequencing reactions.

Suitable detergents for use in such methods include, but are not limited to, anionic detergents such as sodium n-dodecyl sulfate (SDS); and dihydroxy or trihydroxy bile acids (and their salts), such as cholic acid (sodium cholate), deoxycholic acid (sodium deoxycholate), taurodeoxycholic acid (sodium taurodeoxycholate), taurocholic acid (sodium taurocholate), glycodeoxycholic acid (sodium glycodeoxycholate), glycocholic acid (sodium glycocholate); cationic detergents such as cetyl trimethyl-ammonium bromide (CTAB); non-ionic detergents such as the polyoxyethylenes NP-40, TRITON® X-100, TRITON® X114, C₁₂E₈, C₁₂E₉, GENAPOL® X-080, GENAPOL® X-100, LUBROL® PX, BRIJ® 35, TWEEN® 20, and TWEEN® 20; alkyl glycosides such as n-dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, n-hexyl-β-D-glucopyranoside; alkylamine oxides such as lauryl dimethylamine oxide (LDAO); and zwitterionic detergents, such as CHAPS, CHAPSO, n-dodecyl-N,N-dimethylglycine, and ZWITTERGENTS® 3-08, 3-10, 3-12, 3-14, and 3-16.

In yet another aspect, the present invention further provides compositions and kits comprising a purified thermostable DNA polymerase free of any exogenously added detergent, and one or more detergents suitable for addition to the purified DNA polymerase.

### DETAILED DESCRIPTION OF THE PREFERRED EMBODIMENTS

The present invention relates to compositions and methods that permit the skilled artisan to control the environment in which thermostable enzymes, in particular thermostable DNA polymerases, are purified and used. In particular, by purifying thermostable enzymes (e.g., DNA polymerases) in the absence of exogenously added detergents, the skilled artisan may control the timing, identity, and amount of detergent present in any reaction mixture. In this manner, an active enzyme may be provided, while avoiding the presence of detergents that may generate inconsistent or undesirable results under particular conditions.

Purification of Thermostable Enzymes

A variety of procedures have been traditionally employed to facilitate the preparation of intracellular proteins from organisms. As an initial step, the contents of the organism or cells of interest are typically liberated, e.g., by lysis, rupture and/or permeabilization of the cells. Following this release of contents, one or more desired proteins may be purified from the cell extract, often by a series of chromatographic, precipitation, and/or selective binding steps.

Several approaches have proven useful in accomplishing the release of intracellular proteins from cells. Included among these are chemical lysis or permeabilization, physical methods of disruption, or a combination of chemical and physical approaches. Chemical methods of disruption of the bacterial cell wall generally involve treatment of cells with organic solvents, chaotropes, antibiotics, detergents, and/or enzymes. Physical methods generally include osmotic shock, drying, shear forces (employing, for example, bead mills or blenders), temperature shock, ultrasonic disruption, or some combination of the above (e.g., a French press generates both shear forces and an explosive pressure drop). Other approaches combine chemical and physical methods of disruption generally involve lysozyme treatment followed by sonication or pressure treatment to maximize cell disruption and protein release.

As discussed above, detergents are often employed to rapidly disrupt the cell such that the release of intracellular proteins is maximized, and such approaches have been used in the initial steps of processes for the purification of a variety of bacterial cytosolic enzymes, including natural and recombinant proteins from mesophilic organisms such as Escherichia coli, and from thermophilic bacteria and archaea such as those described herein. However, even when detergents are not employed during the initial steps of fractionation, they are often added subsequently in order to facilitate fractionation of the cell extract into various sub-portions.

In order to provide a purified thermostable enzyme composition, the present invention requires that both lysis and purification steps are performed in the absence of exogenously added detergent. Thermostable enzymes that can be prepared and used according to the present invention methods may be obtained from a variety of thermophilic bacteria that are available commercially (for example, from American Type Culture Collection, Rockville, Md.). Suitable for use as sources of thermostable enzymes are the thermophilic bacteria Thermus flavus, Meiothermus ruber, Thermus thermophilus, Bacillus stearothermophilus, Thermus aquaticus, Thermus lacteus, Thermus oshimai, Methanothermus fervidus, Sulfolobus solfataricus, Sulfolobus acidocaldarius, Thermoplasma acidophilum, Methanobacterium thermoautotrophicum and Desulfurococcus mobilis, and other species of the Pyrococcus or Thermotoga genera. It will be understood by one of ordinary skill in the art, however, that any thermophilic microorganism may be used as a source for preparation of thermostable enzymes according to the present invention methods. Additionally, a DNA sequence encoding a thermostable enzyme of interest may be expressed in an organism (e.g., E. coli) that does not normally express such an enzyme, using recombinant DNA methods well known to those of skill in the art. See, e.g., Lu and Erickson, Protein Expr. Purif 11: 179-84 (1997); Desai and Pfaffle, Biotechniques 19: 780-2, 784 (1995).

Particularly preferred thermostable enzymes include those provided in Table 1, together with functional variants thereof. The term “functional variant” refers to polypeptides in which one or more amino acids have been substituted and/or added and/or deleted, but that still retain at least 10% of one or more enzymatic activities (e.g., DNA polymerase activity) performed by the parent thermostable enzyme.

TABLE 1

(SEQ ID NO: 1)

Taq DNA Polymerase (AmpliTaq ™)

| | |
|---|---|
| 1 | mrgmlplfep kgrvllvdgh hlayrtfhal kglttsrgep vqavygfaks llkalkedgd |
| 61 | avivvfdaka psfrheaygg ykagraptpe dfprqlalik elvdllglar levpgyeadd |
| 121 | vlaslakkae kegyevrilt adkdlyqlls drihvlhpeg ylitpawlwe kyglrpdqwa |
| 181 | dyraltgdes dnlpgvkgig ektarkllee wgsleallkn ldrlkpaire kilahmddlk |
| 241 | lswdlakvrt dlplevdfak rrepdrerlr aflerlefgs llhefglles pkaleeapwp |
| 301 | ppegafvgfv lsrkepmwad llalaaargg rvhrapepyk alrdlkearg llakdlsvla |
| 361 | lreglglppg ddpmllayll dpsnttpegv arryggewte eageraalse rlfanlwgrl |
| 421 | egeerllwly reverplsav lahmeatgvr ldvaylrals levaeeiarl eaevfrlagh |
| 481 | pfnlnsrdql ervlfdelgl paigktektg krstsaavle alreahpive kilqyreltk |
| 541 | lkstyidplp dlihprtgrl htrfnqtata tgrlsssdpn lqnipvrtpl gqrirrafia |
| 601 | eegwllvald ysqielrvla hlsgdenhir vfqegrdiht etaswmfgvp reavdplmrr |
| 661 | aaktinfgvl ygmsahrlsq elaipyeeaq afieryfqsf pkvrawiekt leegrrrgyv |
| 721 | etlfgrrryv pdlearvksv reaaermafn mpvqgtaadl mklamvklfp rleemgarml |
| 781 | lqvhdelvle apkeraeava rlakevmegv yplavpleve vgigedwlsa ke |

(SEQ ID NO: 2)

Tth DNA Polymerase

| | |
|---|---|
| 1 | meamlplfep kgrvllvdgh hlayrtffal kglttsrgep vqavygfaks llkalkedgy |
| 61 | kavfvvfdak apsfrheaye aykagraptp edfprqlali kelvdulgft rlevpgyead |
| 121 | dvlatlakka ekegyevril tadrdlyqlv sdrvavlhpe ghlitpewlw ekyglrpeqw |
| 181 | vdfralvgdp sdnlpgvkgi gektalkllk ewgslenllk nldrvkpenv rekikahled |
| 241 | lrlslelsrv rtdlplevdl aqgrepdreg lraflerlef gsllhefgll eapapleeap |
| 301 | wpppegafvg fvlsrpepmw aelkalaacr dgrvhraadp laglkdlkev rgllakdlav |
| 361 | lasregldlv pgddpmllay lldpsnttpe gvarryggew tedaahrall serlhrnllk |
| 421 | rlegeekllw lyhevekpls rvlahmeatg vrrdvaylqa lslelaeeir rleeevfrla |
| 481 | ghpfnlnsrd qlervlfdel rlpalgktqk tgkrstsaav lealreahpi vekilqhrel |
| 541 | tklkntyvdp lpslvhprtg rlhtrfnqta tatgrlsssd pnlqnipvrt plgqrirraf |
| 601 | vaeagwalva ldysqielrv lahlsgdenl irvfqegkdi htqtaswmfg vppeavdplm |
| 661 | rraaktvnfg vlygmsahrl sqelaipyee avafieryfq sfpkvrawie ktleegrkrg |
| 721 | yvetlfgrrr yvpdlnarvk svreaaerma fnmpvqgtaa dlmklamvkl fprlremgar |
| 781 | mllqvhdell leapqaraee vaalakeame kayplavple vevgmgedwl sakg |

(SEQ ID NO: 3)

Thermus oshimai DNA Polymerase (Tsp spsl7)

| | |
|---|---|
| 1 | mlplfepkgr vllvdghhla yrtffalkgl ttsrgepvqa vygfaksllk alkedgevai |
| 61 | vvfdakapsf rheayeayka graptpedfp rqlalikelv dllglvrlev pgfeaddvla |
| 121 | tlakkaereg yevrilsadr dlyqllsdri hllhpegevl tpgwlqeryg lsperwveyr |
| 181 | alvgdpsdnl pgvpgigekt alkllkewgs leailknldq vkpervreai rnnldklqms |
| 241 | lelsrlrtdl plevdfakrr epdweglkaf lerlefgsll hefglleapk eaeeapwppp |
| 301 | ggaflgflls rpepmwaell alagakegrv hraedpvgal kdlkeirgll akdlsvlalr |
| 361 | egreippgdd pmllaylldp gntnpegvar ryggewkeda aarallserl wqalyprvae |
| 421 | eerllwlyre verplaqvla hmeatgvrld vpylealsqe vafelerlea evhrlaghpf |
| 481 | nlnsrdqler vlfdelglpp igktektgkr stsaavlell reahpivgri leyrelmklk |
| 541 | styidplprl vhpktgrlht rfnqtatatg rlsssdpnlq nipvrtplgq rirkafiaee |
| 601 | ghllvaldys qielrvlahl sgdenlirvf regkdihtet aawmfgvppe gvdgamrraa |
| 661 | ktvnfgvlyg msahrlsqel sipyeeaaaf ieryfqsfpk vrawiaktle egrkkgyvet |
| 721 | lfgrrryvpd lnarvksvre aaermafnmp vqgtaadlmk lamvklfprl rplgvrillq |
| 781 | vhdelvleap karaeeaaql aketmegvyp lsvplevevg mgedwlsaka |

(SEQ ID NO: 4)

Pfu DNA Polymerase

| | |
|---|---|
| 1 | mildvdyite egkpvirlfk kengkfkieh drtfrpyiya llrddskiee vkkitgerhg |
| 61 | kivrivdvek vekkflgkpi tvwklylehp qdvptirekv rehpavvdif eydipfakry |
| 121 | lidkglipme geeelkilaf dietlyhege efgkgpiimi syadeneakv itwknidlpy |
| 181 | vevvsserem ikrflriire kdpdiivtyn gdsfdfpyla kraeklgikl tigrdgsepk |
| 241 | mqrigdmtav evkgrihfdl yhvitrtinl ptytleavye aifgkpkekv yadeiakawe |
| 301 | sqenlervak ysmedakaty elgkeflpme iqlsrlvgqp lwdvsrsstg nlvewfllrk |
| 361 | ayernevapn kpseeeyqrr lresytggfv kepekglwen ivyldfraly psiiithnvs |
| 421 | pdtlnlegck nydiapqvgh kfckdipgfi psllghllee rqkiktkmke tqdpiekill |
| 481 | dyrqkaikll ansfygyygy akarwyckec aesvtawgrk yielvwkele ekfgfkvlyi |
| 541 | dtdglyatip ggeseeikkk alefvkyins klpglleley egfykrgffv tkkryavide |
| 601 | egkvitrgle ivrrdwseia ketqarvlet ilkhgdveea vrivkeviqk lanyeippek |
| 661 | laiyeqitrp lheykaigph vavakklaak gvkikpgmvi gyivlrgdgp isnrailaee |
| 721 | ydpkkhkyda eyyienqvlp avlrilegfg yrkedlryqk trqvgltswl nikks |

(SEQ ID NO: 5)

Bst DNA Polymerase

| | |
|---|---|
| 1 | mknklvlidg nsvayraffa lpllhndkgi htnavygftm mlnkilaeeq pthilvafda |
| 61 | gkttfrhetf qdykggrqqt ppelseqfpl lrelikayri payeldhyea ddiigtmaar |
| 121 | aeregfavkv isgdrdltql aspqvtveit kkgitdiesy tpetvvekyg ltpeqivdlk |
| 181 | glmgdksdni pgvpgigekt avkllkqfgt venvlaside ikgeklkenl rqyrdlalls |
| 241 | kqlaaicrda pveltlddiv ykgedrekvv alfqelgfqs fldkmavqtd egekplagmd |
| 301 | faiadsvtde mladkaalvv evvgdnyhha pivgialane rgrfflrpet aladpkflaw |
| 361 | lgdetkkktm fdskraaval kwkgielrgv vfdlllaayl ldpaqaagdv aavakmhqye |
| 421 | avrsdeavyg kgakrtvpde ptlaehlvrk aaaiwaleep lmdelrrneq drllteleqp |
| 481 | lagilanmef tgvkvdtkrl eqmgaelteq lqaverriye lagqefnins pkqlgtvlfd |
| 541 | klqlpvlkkt ktgystsadv leklaphhei vehilhyrql gklqstyieg llkvvhpvtg |
| 601 | kvhtmfnqal tqtgrlssve pnlqnipirl eegrkirqaf vpsepdwlif aadysqielr |
| 661 | vlahiaeddn lieafrrgld ihtktamdif hvseedvtan mrrqakavnf givygisdyg |
| 721 | laqnlnitrk eaaefieryf asfpgvkqym dnivqeakqk gyvttllhrr rylpditsrn |
| 781 | fnvrsfaert amntpiqgsa adiikkamid lsvrlreerl qarlllqvhd elileapkee |
| 841 | ierlcrlvpe vmeqavtlrv plkvdyhygp twydak |

(SEQ ID NO: 6)

Tli DNA Polymerase

| | |
|---|---|
| 1 | mildtdyitk dgkpiirifk kengefkiel dphfqpyiya llkddsaiee ikaikgerhg |
| 61 | ktvrvldavk vrkkflgrev evwklifehp qdvpamrgki rehpavvdiy eydipfakry |
| 121 | lidkglipme gdeelkllaf dietfyhegd efgkgeiimi syadeeearv itwknidlpy |
| 181 | vdvvsnerem ikrfvqvvke kdpdviityn gdnfdlpyli kraeklgvrl vlgrdkehpe |
| 241 | pkiqrmgdsf aveikgrihf dlfpvvrrti nlptytleav yeavlgktks klgaeeiaai |
| 301 | weteesmkkl aqysmedara tyelgkeffp meaelaklig qsvwdvsrss tgnlvewyll |
| 361 | rvayarnela pnkpdeeeyk rrlrttylgg yvkepekglw eniiyldfrs lypsiivthn |
| 421 | vspdtlekeg cknydvapiv gyrfckdfpg fipsilgdli amrqdikkkm kstidpiekk |
| 481 | mldyrqraik llansyygym gypkarwysk ecaesvtawg rhyiemtire ieekfgfkvl |
| 541 | yadtdgfyat ipgekpelik kkakeflnyi nsklpgllel eyegfylrgf fvtkkryavi |
| 601 | deegrittrg levvrrdwse iaketqakvl eailkegsve kavevvrdvv ekiakyrvpl |
| 661 | eklviheqit rdlkdykaig phvaiakrla argikvkpgt iisyivlkgs gkisdrvill |
| 721 | teydprkhky dpdyyienqv lpavlrilea fgyrkedlry qsskqtglda wlkr |

(SEQ ID NO: 7)

KOD DNA Polymerase

| | |
|---|---|
| 1 | mildtdyite dgkpvirifk kengefkiey drtfepyfya llkddsaiee vkkitaerhg |
| 61 | tvvtvkrvek vqkkflgrpv evwklyfthp qdvpairdki rehpavidiy eydipfakry |
| 121 | lidkglvpme gdeelkmlaf dietlyhege efaegpilmi syadeegarv itwknvdlpy |
| 181 | vdvvsterem ikrflrvvke kdpdvlityn gdnfdfaylk krceklginf algrdgsepk |
| 241 | iqrmgdrfav evkgrihfdl ypvirrtinl ptytleavye avfgqpkekv yaeeittawe |
| 301 | tgenlervar ysmedakvty elgkeflpme aqlsrliggs lwdvsrsstg nlvewfllrk |
| 361 | ayernelapn kpdekelarr rqsyeggyvk eperglweni vyldfr |
| 421 | |
| 481 | |
| 541 | |
| 601 | |
| 661 | |
| 721 | slyp siiithnvsp |
| 781 | dtlnregcke ydvapqvghr fckdfpgfip sllgdlleer qkikkkmkat idpierklld |
| 841 | yrqraikila n |
| 901 | |
| 961 | |
| 1021 | |
| 1081 | |
| 1141 | |
| 1201 | |
| 1261 | |
| 1321 | |
| 1381 | sy ygyygyarar wyckecaesv tawgreyitm tikeieekyg fkviysdtdg |
| 1441 | ffatipgada etvkkkamef lkyinaklpg aleleyegfy krgffvtkkk yavideegki |
| 1501 | ttrgleivrr dwseiaketq arvleallkd gdvekavriv kevteklsky evppeklvih |
| 1561 | eqitrdlkdy katgphvava krlaargvki rpgtvisyiv lkgsgrigdr aipfdefdpt |
| 1621 | khkydaeyyi enqvlpaver ilrafgyrke dlryqktrqv glsawlkpkg t |

Note: for clarity, the expressed protein amino acid numbering in the
foregoing is preserved, but the two intervening sequences (inteins)
have been removed as they would be in active enzyme. See, Perler, FB,
Nucleic Acids Res. 2002 Jan 1;30(1):383-4.

(SEQ ID NO: 8)

NTba DNA Polymerase

| | |
|---|---|
| 1 | mildvdyite dgkpvirvfk kdkgefkiey drefepyiya llrddsaiee iekitaerhg |
| 61 | kvvkvkraek vkkkflgrsv evwvlyfthp qdvpairpdk irkhpavidi yeydipfakr |
| 121 | ylidkglipm egdeelklms fdietlyheg eefgtgpilm isyadesear vitwkkidlp |
| 181 | yvdvvsteke mikrflkvvk ekdpdvlity dgdnfdfayl kkrceklgvs ftlgrdgsep |
| 241 | kiqrmgdrfa vevkgrihfd lypairrtin lptytleavy eavfgkpkek vyaeeiataw |
| 301 | etgeglegva rysmedarvt yelgreffpm eaqlsrligq glwdvsrsst gnlvewfllr |
| 361 | kayernelap nkpderelar rrggyaggyv keperglwdn ivyldfrsly psiiithnvs |
| 421 | pdtlnregck sydvapqvgh kfckdfpgfi psllgnllee rqkikrkmka tldplerkll |
| 481 | dyrqraikil ansfygyygy ararwyckec aesvtawgre yiemvirele ekfgfkdlya |
| 541 | dtdglhatip gadretvkkk dleflnyinp klpglleley egfysrgffv tkkkyavide |
| 601 | egkittrgle ivrrdwseia ketlarvlea ilrhgdveea vrivkeetek lskyevppek |
| 661 | lviteqitre lkdykatgph vaiakrlaar gikirpgtvi syivlkgsgr igdraipfde |
| 721 | fdptkhryda dyyienqvlp averilrafg ykkederyqk trqvglgawl gmggerlkl |

(SEQ ID NO: 9)

Tba DNA Polymerase

| | |
|---|---|
| 1 | mildvdyite dgkpvirvfk kdkgefkiey drefepyiya llrddsaiee iekitaerhg |
| 61 | kvvkvkraek vkkkflgrsv evwvlyfthp qdvpairpdk irkhpavidi yeydipfakr |
| 121 | ylidkglipm egdeelklms fdietlyheg eefgtgpilm isyadesear vitwkkidlp |
| 181 | yvdvvsteke mikrflkvvk ekdpdvlity dgdnfdfayl kkrceklgvs ftlgrdgsep |
| 241 | kiqrmgdrfa vevkgrihfd lypairrtin lptytleavy eavfgkpkek vyaeeiataw |
| 301 | etgeglegva rysmedarvt yelgreffpm eaqlsrligq glwdvsrsst gnlvewfllr |
| 361 | kayernelap nkpderelar rrggyaggyv keperglwdn ivyldfrsly psiiithnvs |
| 421 | pdtlnregck sydvapqvgh kfckdfpgfi psllgnllee rqkikrkmka tldplerkll |
| 481 | dyrqraikil ansfygyygy ararwyckec aesvtawgre yiemvirele ekfgfkdlya |
| 541 | dtdglhatip gadretvkkk dleflnyinp klpglleley egfysrgffv tkkkyavide |
| 601 | egkittrgle ivrrdwseia ketlarvlea ilrhgdveea vrivkeetek lskyevppek |
| 661 | lviteqitre lkdykatgph vaiakrlaar gikirpgtvi syivlkgsgr igdraipfde |
| 721 | fdptkhryda dyyienqvlp averilrafg ykkederyqk trqvglgawl gmgqerlkl |

(SEQ ID NO: 10)

Taq Δ271 F667Y DNA Polymerase (Thermo Sequenase ™)

| | |
|---|---|
| 1 | |
| 61 | |
| 121 | |
| 241 | mlerlefgs llhefglles pkaleeapwp |
| 301 | ppegafvgfv lsrkepmwad llalaaargg rvhrapepyk alrdlkearg llakdlsvla |
| 361 | lreglglppg ddpmllayll dpsnttpegv arrygqewte eageraalse rlfanlwgrl |
| 421 | egeerllwly reverplsav lahmeatgvr ldvaylrals levaeeiarl eaevfrlagh |
| 481 | pfnlnsrdql ervlfdelgl paigktektg krstsaavle alreahpive kilqyreltk |
| 541 | lkstyidplp dlihprtgrl htrfnqtata tgrlsssdpn lqnipvrtpl gqrirrafia |
| 601 | eegwllvald ysqielrvla hlsgdenlir vfqegrdiht etaswmfgvp reavdplmrr |
| 661 | aaktinygvl ygmsahrlsq elaipyeeaq afieryfqsf pkvrawiekt leegrrrgyv |
| 721 | etlfgrrryv pdlearvksv reaaermafn mpvqgtaadl mklamvklfp rleemgarml |
| 781 | lqvhdelvle apkeraeava rlakevmegv yplavpleve vgigedwlsa ke |

(SEQ ID NO: 11)

Tth Δ273 F668Y DNA Polymerase

| | |
|---|---|
| 1 | |
| 61 | |
| 121 | |
| 241 | mlerlef gsllhefgll eapapleeap |
| 301 | wpppegafvg fvlsrpepmw aelkalaacr dgrvhraadp laglkdlkev rgllakdlav |
| 361 | lasregldlv pgddpmllay lldpsnttpe gvarryggew tedaahrall serlhrnllk |
| 421 | rlegeekllw lyhevekpls rvlahmeatg vrrdvaylqa lslelaeeir rleeevfrla |
| 481 | ghpfnlnsrd qlervlfdel rlpalgktqk tgkrstsaav lealreahpi vekilqhrel |
| 541 | tklkntyvdp lpslvhprtg rlhtrfnqta tatgrlsssd pnlqnipvrt plgqrirraf |
| 601 | vaeagwalva ldysqielrv lahlsgdenl irvfqegkdi htqtaswmfg vppeavdplm |
| 661 | rraaktvnyg vlygmsahrl sqelaipyee avafieryfq sfpkvrawie ktleegrkrg |
| 721 | yvetlfgrrr yvpdlnarvk svreaaerma fnmpvqgtaa dhnklamvkl fprlremgar |
| 781 | mllqvhdell leapqaraee vaalakeame kayplavple vevgmgedwl sakg |

(SEQ ID NO: 12)

Taq Δ271 F667Y E681W DNA Polymerase

| | |
|---|---|
| 1 | |
| 61 | |
| 121 | |
| 241 | mlerlefgs llhefglles pkaleeapwp |
| 301 | ppegafvgfv lsrkepmwad llalaaargg rvhrapepyk alrdlkearg llakdlsvla |
| 361 | lreglglppg ddpmllayll dpsnttpegv arryggewte eageraalse rlfanlwgrl |
| 421 | egeerllwly reverplsav lahmeatgvr ldvaylrals levaeeiarl eaevfrlagh |
| 481 | pfnlnsrdql ervlfdelgl paigktektg krstsaavle alreahpive kilqyreltk |
| 541 | lkstyidplp dlihprtgrl htrfnqtata tgrlsssdpn lqnipvrtpl gqrirrafia |
| 601 | eegwllvald ysqielrvla hlsgdenlir vfqegrdiht etaswmfgvp reavdplmrr |
| 661 | aaktinygvl ygmsahrlsq wlaipyeeaq afieryfqsf pkvrawiekt leegrrrgyv |
| 721 | etlfgrrryv pdlearvksv reaaermafn mpvqgtaadl mklamvklfp rleemgarml |
| 781 | lqvhdelvle apkeraeava rlakevmegv yplavpleve vgigedwlsa ke |

In various embodiments of the present invention, procedures may be designed for purification of the enzyme(s) without using any exogenously added detergent, and the activity of the purified enzyme may be examined using standard activity assays. The purification procedure generally contains the following steps.

Stock reagents and purification buffers (which do not contain any detergents) are prepared, and a cell suspension or pellet is subjected to disruption, e.g., using a French press, nitrogen “bomb” disruptor, or shear forces, to obtain a lysate containing the enzyme(s) of interest. This lysate is then subjected to one or more purification procedures.

Protein purification procedures are well known to those of skill in the art. See, e.g., Deutscher, Methods in Enzymology, Vol. 182, “Guide to Protein Purification,” 1990. Various precipitation, chromatographic, and/or electrophoretic methods may be employed to purify the enzyme(s) of interest from the lysate. These include precipitation by various means (e.g., using ammonium sulfate or polycations such as polyethylenimine), ion exchange chromatography (e.g., using DEAE, quarternary amine, phosphoryl and/or carboxyl functionalities on cellulose, agarose or polymeric beads), affinity chromatography (e.g., heparin on agarose or polymeric beads), hydrophobic interaction chromatography (e.g., butyl, octyl, phenyl or hexyl functionalities on agarose or polymeric beads), hydroxylapatite chromatography, size exclusion chromatography, etc. Chromatography may be performed using low pressures (e.g., gravity-driven flow), or at higher pressures (e.g., using instruments with pumps such as FPLC or HPLC).

Additionally, one can take advantage of the thermostability of the enzymes of interest by using heat treatment as a separation step. Many proteins that are not thermostable are denatured, and thereby precipitated, while thermostable enzymes will often be less susceptible to denaturation by heat. Preferably, a heat treatment step is performed at a temperature between about 50° C. and 95° C., more preferably between about 65° C. and 85° C.; and most preferably between about 70° C. and 80° C. for between about 5 minutes and about 5 hours, more preferably for between about 15 minutes and about 2 hours, and most preferably for less than or equal to about 1 hour. The term “about” in this context refers to +/− 10% of a given measurement. Denatured proteins may be removed, e.g., by centrifugation, and the remaining material used for further processing.

Uses of Thermostable DNA Polymerases

Once obtained, the purified thermostable enzymes of the present invention may be used in standard methods well known to those of skill in the art. With regard specifically to DNA polymerases (e.g., those described in the previous “purification” section), such methods include but are not limited to DNA polymerase activity reactions, DNA sequencing reactions, amplification reactions such as PCR, single-stranded endonuclease reactions, double-stranded endonuclease reactions, single-stranded exonuclease reactions and double-stranded exonuclease reactions. See, e.g., Lawyer et al., J. Biol. Chem. 1989 Apr 15;264(11):6427-37; Kong et al., J. Biol. Chem. 1993 Jan 25;268(3):1965-75; Tabor and Richardson, J. Biol. Chem. 1989 Apr 15;264(11):6447-58; and Lyamichev et al., Proc. Natl. Acad. Sci. U. S. A. 1999 May 25;96(11):6143-8. Particularly preferred are DNA sequencing methods, most preferably dideoxy chain termination sequencing methods. See, e.g., Roe, Crabtree and Khan, “DNA Isolation and Sequencing” (Essential Techniques Series), John Wiley & Sons, 1996; Graham and Hill, Eds., DNA Sequencing Protocols, 2^{nd} Ed., Humana Press, 2001.

Certain thermostable DNA polymerases, when purified in the absence of detergents as described herein, will perform poorly in such assays, particularly in dilute solutions. Surprisingly, it has been determined that activity of such enzymes can often be stabilized, restored or enhanced by the addition of one or more detergents to purified thermostable DNA polymerase compositions lacking exogenous detergent. Thus, in various embodiments, the present invention describes the addition of one or more detergents to such compositions, particularly detergents based on poly(ethylene oxide)s, alkyl glycosides, and alkyl amine N-oxides. In addition, protein hydrolysates (e.g., Prionex, a hydrolyzed modified porcine collagen), either alone or in combination with one or more detergents, can also advantageously restore or enhance activity of such enzymes.

Particularly preferred poly(ethylene oxide) detergents have the following formulas, and include NP-40, TRITON® X-100, TRITON® X114, C₁₂E₈, C₁₂E₉, GENAPOL® X-080,

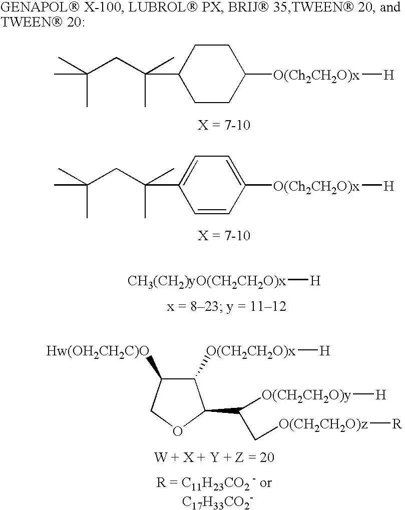

Preferred alkyl glycosides have the following formulas, and include n-dodecyl-β-D-maltoside (“dodecyl maltoside”), n-nonyl-β-D-glucopyranoside, n-octyl-β-D-glucopyranoside (“octyl glucoside”), n-heptyl-β-D-glucopyranoside, n-hexyl-β-D-glucopyranoside, and octyl-β-D-thioglucopyranoside:

R—O—(CH₂)ₓ—CH₃ R=glucose, maltose, lactose, xylose, galactose, x=5-16;

R—S—(CH₂)ₓ—CH₃ R=glucose, maltose, lactose, xylose, galactose, x=5-16

Preferred alkyl amine N-oxides have the following formula and include lauryl dimethylamine oxide:

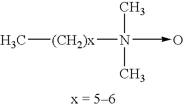

It will be readily apparent to those skilled in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### Purification of DNA Polymerase

This example describes a process to purify thermophilic DNA polymerase from a frozen bacterial cell paste.

Reagent and Buffer Preparation

Lysis buffer was prepared by mixing Tris HCl (pH 8.5), EDTA and ammonium sulfate. The final concentration for Tris HCl, EDTA and ammonium sulfate in the buffer solution was 50 mM, 2 mM, and 1 M, respectively. The pH of this buffer solution was adjusted to 8.5±0.1 at room temperature. The buffer was stored at 4° C. for up to one week, and was filtered before use.

100 mMPMSF: 1 g PMSF was added to 60 ml of isopropanol in an appropriate container, vortexed to mix thoroughly (this material does not go into solution very easily). The solution was stored at 4° C. for one month. Heat gently (<50° C.) to re-dissolve any material that crystallizes out during storage prior to use.

Buffer A was prepared by mixing Tris HCl (pH 8.5), EDTA, ammonium sulfate, and DTT. The final concentration for Tris HCl, EDTA, ammonium sulfate and DTT was 50 mM, 1 mM, 1M, and 1 mM, respectively. The pH for buffer A was adjusted to 8.5±0.1 at room temperature with HCl (6N). Buffer A was used for equilibrating butyl Sepharose FF column.

Buffer B was prepared by mixing Tris HCl (pH 8.5), EDTA, and DTT. The final concentration for Tris HCl, EDTA, and DTT was 50 mM, 1 mM, and 1 mM, respectively. The pH for buffer B was adjusted to 8.5±0.1 at room temperature with HCl (6N). Buffer B was also used for Butyl Sepharose FF column. Both Buffer A and B were sterile filtered, and stored at 4° C. for up to one week.

Final dialysis buffer with glycerol: The final dialysis buffer was prepared by mixing solutions of Tris HCl, EDTA, and KCl with glycerol and H₂O. The final concentration for Tris HCl, EDTA and KCl was 20 mM, 0.1 mM, and 25 mM, respectively. The final concentration of glycerol was 50% (v/v). The pH of the buffer was adjusted to 8.5±0.1 at room temperature with 6N HCl. The buffer must be autoclaved before use (do not filter), and then DTT added (final concentration was 1 mM) to the buffer after the buffer is autoclaved and cooled down to 4° C.

TABLE 2
Butyl Sepharose FF BPG 140/500 column preparation for purification

| | |
|---|---|
| Bed volume | 1500 ml packed |
| Column type (or | BPG140/500 |
| equivalent) | |
| Equilibrate with | 3 Column Volumes (CV) Buffer A |
| Flow Rate | 75 ml/min |
| Load Sample with | Pump A18 |
| After sample is loaded, | 10 CV |
| wash with | |
| Elution | 0-40% B in 1CV, hold at 40% for 5CV (or |
| | until A260/A280 nm returns to baseline) |
| | 40-70% in 3CV, hold at 70% for 5CV (or |
| | until A260/A280 nm returns to baseline), |
| Start collection | At 40% B |
| Fraction size | 100 ml (total peak volume should be 4-6 L) |

Column equilibration with butyl sepharose buffer A was at 75 ml/min (30 cm/h, column cross sectional area is 154 cm²) at system pressure of 2.0 bar or less (this is 75% of packing pressure of 2.7 bar). Column equilibration was monitored by inline conductivity and was achieved once a stable reading was reached. Typically, 2 column volumes(CV) should prove adequate for equilibration. Column performance was monitored by injecting 1% of total CV of 1.5% acetone in buffer A at 15 cm/h. Assymetry is between 0.85-1.6, HETP is 0.018-0.036 cm with 2800-5500 N/m.

Bacterial Cell Lysis:

A paste of E. coli expressing a recombinant thermostable DNA polymerase was transferred from a −80° C. freezer to 4° C. on the day before bacterial cell lysis. The pre-chilled lysis buffer was added to the cells (5 ml/g), followed immediately by adding PMSF (100 mM), and mixed continuously until homogenous. The large volume of sample may be divided for the lysis step, provided that the other portion of the sample is kept at 4° C. until it can be lysed. The press was pre-chilled to 4° C. and flushed with 200-500 mls of 4° C. lysis buffer. Once the cell paste was evenly resuspended, the cells were passed through the press at 12-15,000 PSIG. Lysate was collected when the outlet-line on press became cloudy/milky. Lysate was slightly viscous. This was passed through the press a second time under same conditions without further priming. Lysate after second pass was no longer viscous.

Heat Precipitation

The container of lysed cells was placed into a pre-heated water bath at 85±2° C. for denaturation. The temperature of the lysate was monitored with a thermometer placed in the lysate. Once the temperature reached 75±2° C., the sample was incubated for 40 min. After 40 min, the sample was removed and placed immediately on ice with gentle swirling for cooling down to <10° C. The cooled cells were distributed into 1 L bottles. A small sample (<200 μl) of the cell extract was saved for later estimate of sample yield.

The cell extract was then centrifuged at 8,000 rpm in a Beckman JLA 8.100 rotor at 4° C. for 30 min (rcf=16,000). The supernatant was poured into a clean container, and stored in cold room overnight. The cell pellet was discarded. The overnight supernatant was then centrifuged again at 8,000 rpm at 4° C. for 30 min. The clarified cell extract supernatant was collected for later loading onto the butyl sepharose FF column for purification. A small sample (<200 μl) of the clarified cell extract was saved for later purification sample yield estimate.

Butyl Sepharose FF Column Purification

Before loading the clarified cell extract onto the butyl sepharose FF column, the column was flushed with Buffer A. The conductivity and pH of butyl sepharose column effluent were checked and adjusted. The conductivity should be ±10% and pH should be ±0.3 pH of butyl sepharose buffer A. The conductivity of clarified cell extract was also measured. It should be within 10% of butyl sepharose buffer A. No adjustment should be necessary.

The sample was loaded onto the butyl sepharose FF column at 75 ml/min. The non-binding fraction was collected as soon as A(260/280 nm) begins to increase. The column was washed with 10 CV, and eluted with the following gradient: 0-40% in 1 CV; hold at 40% for 5CV or until A(260/280 nm) returns to baseline; 40-70% in 3CV; hold at 70% for 5CV or until A(260/280 nm) returns to baseline; 70-100% in 1CV, hold at 100% for 3CV. Sample collection was begun when the A280 increased. The fractions were stored overnight at 4° C.

The protein that does not bind to the column, the peak fractions, a set of standards, the material loaded onto the column and reference DNA polymerase samples were run in an 8-25% SDS gel. The chromatograph and data including electrophoresis results are recorded.

Sample Dialysis

The sample was then prepared for dialysis. If pooled butyl fraction has any precipitated material, filter before diafiltration. Diafiltration was also used to concentrate the fraction containing DNA polymerase. Once the sample volume is less than 1 L, the sample was placed in dialysis tubing and dialyzed against 3 L of final buffer with glycerol overnight. Buffer was changed at the end of the day and again in the morning of the next day. The DNA polymerase was harvested from dialysis.

In one embodiment of the present invention, Taq Δ271 F667Y, and Taq Δ271 F667Y E68 1W were purified with or without detergents NP-40 & Tween-20. The butyl Sepharose chromatography elution profile for polymerase extracted without detergents was essentially identical to the profile for polymerase extracted with Tween 20 and NP-40. The yield relative to starting material of these enzymes from purification with and without detergents is shown in Tables 3 and 4. The yield of the purified enzymes without the detergents is not significantly different from the yield of the purified enzyme obtained with the detergents.

TABLE 3

| | Detergent present during | Overall |
|---|---|---|
| Enzyme | purification | Yield* |
| Taq Δ271, F667Y | 0.1% Tween 20, 0.1% NP-40 | 130% |
| Taq Δ271, F667Y | None | 111% |
| Taq Δ271, F667Y, E681W | 0.1% Tween 20, 0.1% NP-40 | 118% |
| Taq Δ271, F667Y, E681W | None | 102% |

*% of activity in crude extract assayed under standard conditions.

TABLE 4

| | Detergent in | Detergent | |
|---|---|---|---|
| Enzyme | Purification | in Assay | Assay (%*) |
| Taq Δ271, F667Y | None | None | 5% |
| Taq Δ271, F667Y | None | 0.1% Tween 20, | 102% |
| | | 0.1% NP-40 | |
| Taq Δ271, F667Y | 0.1% Tween 20, | None | 3% |
| | 0.1% NP-40 | | |
| Taq Δ271, F667Y | 0.1% Tween 20, | 0.1% Tween 20, | 100% |
| | 0.1% NP-40 | 0.1% NP-40 | |
| Taq Δ271, F667Y, | None | None | 6% |
| E681W | | | |
| Taq Δ271, F667Y, | None | 0.1% Tween 20, | 157% |
| E681W | | 0.1% NP-40 | |
| Taq Δ271, F667Y, | 0.1% Tween20, | None | 2% |
| E681W | 0.1% NP-40 | | |
| Taq Δ271, F667Y, | 0.1% Tween 20, | 0.1% Tween 20, | 100% |
| E681W | 0.1% NP-40 | 0.1% NP-40 | |

*100% is the specific activity (units/mg protein) of polymerase purified and assayed using Tween 20 and NP-40

### EXAMPLE 2

### Enzyme Activity Assays

DNA polymerase activity was measured by running reactions of 50 μL containing 25 mM TAPS (tris-hydroxymethyl-methylaminopropane sulfonic acid) buffer, pH 9.3 (measured at 25° C.), 50 mM KCl, 2 mM MgCl₂, 1 mM 2-mercaptoethanol, 0.2 mM each of dGTP, dCTP, dTTP, 0.2 mM [α-³³P]-dATP (0.05-0.1 Ci/mmol) and 0.4 mg/mL activated salmon sperm DNA. The reaction mixture (45 μL) was pre-heated to 74° C. and diluted polymerase (5 μL) added with thorough mixing. After 10 minutes of further incubation at 74° C., the reaction was stopped by the addition of 10 μL of 60 mM EDTA and the entire mixture placed at 0° C. Acid-precipitable radioactivity was determined on an aliquot (50 mL) by diluting with 1 ml of 2 mM EDTA containing 0.05 mg/ml salmon sperm DNA and adding 1 mL of 20% (w/v) trichloroacetic acid, 2% (w/v) sodium pyrophosphate (Na₄P₂O₇·10H₂O) and incubating on ice for at least 15 minutes. Precipitated DNA was collected by filtering through 2.4 cm GFC filter disks (Schleichter and Schuell) and washed 7 times with 5ml of with 1 N HCl, 0.1 M sodium pyrophosphate. The filter was placed in 3 ml of aqueous scintillation counting fluid and ³³P-specific radioactivity determined by scintillation counting.

For the assays presented in Tables 5 and 6, the polymerase was diluted 10-5000 fold in a buffer containing 25 mM Tris-HCl pH 8.0, 50 mM KCl, 1 mM 2-mercaptoethanol, and the indicated concentration of detergent or other additive. Where possible, only reactions which incorporated 20-100 pmol of dAMP in 10 minutes were used for calculation of activity.

TABLE 5

| | Concentration | Polymerase A | Polymerase B | Polymerase C |
|---|---|---|---|---|
| Detergent | % (w/v) | Activity (%) | Activity (%) | Activity (%) |
| Tween-20 & NP-40 | 0.5% each | 100 | 100 | 100 |
| Dodecyl Maltoside | 0.01% | 98.8 | 92.3 | 80.8 |
| Mega-8 (glucamide) | 0.5% | 76.6 | 71 | 84.5 |
| Mega-9 | 0.05% | 71.2 | 82 | 74 |
| Mega-10 | 0.05% | 94 | 73 | 100 |
| Lauryl dimethylamine | 0.01% | 1 | 93 | 80.6 |
| oxide (LDAO) | | | | |
| Dodecyl Maltoside & | 0.01%, 0.1% | — | 99 | 83.1 |
| Prionex | | | | |
| LDAO & Prionex | 0.01%, 0.1% | — | 89.2 | 87 |
| Octyl Glucopyranoside | 0.1% | — | 1 | 79.7 |
| None | | 1 | 1 | 1 |

It has been demonstrated that detergents NP-40 & Tween-20, while not present during purification, but present during activity assay, provided active forms of Taq Δ271 F667Y (polymerase A), Taq Δ271 F667Y E681W (polymerase B) and Tth Δ273 F668Y (polymerase C) activities in the desired reactions and assays. Other detergents and compounds were also demonstrated to be suitable for diluting and increasing the polymerase activities in an assay reaction mixture. Since different detergent can increase different polymerase activities, such detergents may be useful in an assay to differentiate the different activities of different polymerases.

TABLE 6

| | Final | | | Taq Δ271 F667Y |
|---|---|---|---|---|
| Additive | Concentration* | Taq Δ271 F667Y | Tth Δ273 F668Y | E681W |
| Betaine | 0.1% | | | −−− |
| n-Dodecyl-β-D-Maltoside | 0.001 | | | + |
| | 0.01 | +++ | +++ | +++ |
| | 0.02 | | | + |
| | 0.1 | | | + |
| n-Dodecyl-β-D-Maltoside + glycerol | 0.01% + 5%(v/v) | | | + |
| n-Dodecyl-β-D-Maltoside + Prionex | 0.01% + 0.05% | | | +++ |
| n-Dodecyl-β-D-Maltoside + LDAO | 0.01 + 0.03 | | | −− |
| n-Dodecyl-β-D-Maltoside + Ectoin | 0.01 + 0.01 | | | + |
| Lauryldimethylamine oxide | 0.001 | | | −−− |
| (LDAO) | 0.01 | +++ | +++ | +++ |
| | 0.03 | +++ | +++ | −− |
| LDAO + Prionex | 0.01 + 0.1%(v/v) | | | +++ |
| Mega-10 | 0.05 | ++ | −− | +++ |
| (D-decanoyl-N-methyl | 0.01 | −− | ++ | −−− |
| glucamide) | 0.001 | −−− | −− | |
| Mega-8 | 0.001 | | | −−− |
| (Octanoyl-N-mehtylglucamide) | 0.01 | | | −−− |
| | 0.1 | ++ | ++ | − |
| | 0.5 | −−− | + | +++ |
| | 0.85 | + | + | −− |
| N-octyl β-D-galactopyranoside | 0.001 | −−− | −− | |
| | 0.01 | −−− | −− | −−− |
| | 0.05 | −− | +++ | |
| | 0.1 | | | − |
| | 0.25 | | | + |
| | 0.5 | | | −−− |
| n-octyl-β-D-Galactopyranoside + Prionex | 0.5% + 0.1%(v/v) | | | −−− |
| Prionex | 60 μl/ml | −−− | + | |
| Prionex, boiled | 60 μl/ml | | −− | |
| n-octyl-β-D-Glucopyranoside | 0.1 | −− | +++ | +++ |
| | 0.01 | −−− | −−− | −−− |
| Ectoin | 0.001 | −−− | −−− | −−− |
| | 0.01 | −−− | −−− | −−− |
| | 0.1 | −−− | −−− | −−− |
| E. coli Single-Stranded DNA | 100 μg/ml | | −−− | |
| Binding Protein | 20 μg/ml | | −−− | |
| T4 gene 32 Protein | 100 μg/ml | | −−− | |
| | 20 μg/ml | | −−− | |
| Zwittergent 3-14 | 0.01% | | | −−− |
| Bovine Serum Albumin (BSA) | 60 μg/ml | − | −− | |
| BSA + sucrose | 50 μg/ml + 20% | | −− | |
| BSA + sucrose Block o/n | 500 μg/ml | −−− | −−− | |
| Cysteine | 0.1 | −− | −−− | − |
| gelatin | 50 μg/ml | | −−− | |
| Mega-9 (Nonyl-N- | 0.05% | −− | +++ | ++ |
| methylglucamide) | 0.01% | −−− | −− | −−− |
| Hydroxyectoin | 0.05% | −−− | −−− | −−− |
| | 0.01% | −−− | −−− | −−− |
| glycerol | 1.0% (v/v) | | −−− | |
| 2-Butoxyethanol | 0.1% (v/v) | −−− | −−− | −−− |
| | 0.01% (v/v) | −−− | −−− | −−− |
| 2-Propoxyethanol | 0.1% (v/v) | −−− | −−− | −−− |
| | 0.01% (v/v) | −−− | −−− | −−− |
| 2-(2-Ethylhexyloxy) Ethanol | 0.1% (v/v) | −−− | −−− | −−− |
| | 0.01% (v/v) | −−− | −−− | −−− |
| CHAPS (3-[(3-Cholamido | 0.1 | + | −− | − |
| propyl)dimethylammonio]-1- | 0.05 | − | −− | −− |
| propanesulfonate) | 0.01 | −− | − | −−− |
| CHAPSO (3-[(3-Cholamido | 0.1 | + | −− | −− |
| propyl)dimethylammonio]-2- | 0.05 | −− | −− | −− |
| hydroxy-1-propanesulfonate) | 0.01 | −−− | − | −−− |
| Sodium Cholate | 0.1 | −−− | −−− | −−− |
| | 0.05 | −−− | −−− | −−− |
| | 0.01 | −−− | −−− | −−− |
| Sodium Deoxycholate | 0.1 | −−− | −−− | −−− |
| | 0.05 | −−− | −−− | −−− |
| | 0.01 | −−− | −−− | −−− |
| Zwittergent 3-08 | 0.2 | −− | + | −− |
| | 0.1 | −− | − | −− |
| | 0.05 | −−− | −− | −−− |
| Zwittergent 3-10 | 0.2 | − | + | −− |
| | 0.1 | −− | + | −− |
| | 0.05 | −− | −− | −−− |

*Concentrations expressed as % (w/v) in the final polymerase assay reaction mixture unless specified otherwise.
+++ Activity >80% (relative to activity using 0.5% each NP-40 and Tween 20)
++ Activity 70-80%
+ Activity 60-70%
− Activity 50-60%
−− Activity 20-50%
−−− Activity <20%

Having now fully described the present invention it will be understood by those of ordinary skill in the art that the same can be performed within a wide and equivalent range of conditions, formulations and other parameters without affecting the scope of the invention or any embodiment thereof.

All publications, patents and patent applications cited herein are indicative of the level of skill of those skilled in the art to which this invention pertains, and are herein incorporated by reference in their entirety.

1. A composition comprising a substantially purified thermostable DNA polymerase, wherein said composition lacks exogenously added detergent.

2. The composition of claim 1, wherein the thermostable DNA polymerase is obtained or derived from an organism having a genus selected from the group consisting of Thermus, Pyrococcus, Thermcoccus, Aquifex, Sulfolobus, and Thermotoga.

3. The composition of claim 1 wherein said DNA polymerase is selected from the group consisting of Taq DNA polymerase, Tth DNA polymerase, Pfu DNA polymerase, Bst DNA polymerase, Tli DNA polymerase, KOD DNA polymerase, nTha DNA polymerase, Tha DNA polymerase, Taq Δ271 F667Y, Tth Δ273 F668Y, and Taq Δ271 F667Y E681W.

4. A method of substantially purifying a thermostable DNA polymerase from cells, comprising:
(a) lysing said cells in the absence of exogenously added detergent to provide a lysate; and
(b) performing one or more purification steps in the absence of exogenously added detergent, whereby a substantially purified thermostable DNA polymerase is obtained from said lysate, and wherein said substantially purified thermostable DNA polymerase is free of exogenously added detergent.

5. The method of claim 4, wherein said purification steps performed in the absence of exogenously added detergent comprise:
(a) heating said lysate to denature one or more proteins;
(b) centrifuging said lysate and removing all or a portion of the supernatant to provide a clarified lysate; and
(c) fractionating said clarified lysate using a chromatography medium comprising a butyl functionality.

6. The method of claim 4, wherein the thermostable DNA polymerase is obtained or derived from an organism having a species selected from the group consisting of Thermus, Pyrococcus, Thermococcus, Thermococcus, Aquifex, Sulfolobus, and Thermotoga.

7. The method of claim 4, wherein said DNA polymerase is selected from the group consisting of Taq DNA polymerase, Tth DNA polymerase, Pfu DNA polymerase, Bst DNA polymerase, Tli DNA polymerase, KOD DNA polymerase, nTha DNA polymerase, Tha DNA polymerase, Taq Δ271 F667Y, Tth Δ273 F668Y, and Taq Δ271 F667Y E681W.

8. A method to provide a purified thermostable DNA polymerase of interest in an active form in an assay, comprising;
adding one or more detergents to a purified thermostable DNA polymerase composition that is free of exogenously added detergent.

9. The method of claim 8 wherein said one or more detergents are selected from the group consisting of Tween 20, Iconol NP-40, Mega-8, Mega-9, Mega-10, alkyl glycosides, and alkyl tertiary amine N-oxides.

10. The method of claim 9 wherein said alkyl glycosides are selected from the group consisting of octyl-beta-D-glucopyranoside and dodecyl-beta-D-maltoside.

11. The method of claim 9 wherein alkyl tertiary amine N-oxide is lauryl dimethyl amine oxide (LDAO).

12. The method of claim 8 wherein said DNA polymerase is selected from the group consisting of Taq DNA polymerase, Tth DNA polymerase, Pfu DNA polymerase, Bst DNA polymerase, Tli DNA polymerase, KOD DNA polymerase, nTha DNA polymerase, Tha DNA polymerase, Taq Δ271 F667Y, Tth Δ273 F668Y, and Taq Δ271 F667Y E681W.

13. The method of claim 8 wherein said DNA polymerase is provided in an active form to a sequencing reaction.

14. The method of claim 8 wherein said assay is selected from the group consisting of thermostable DNA polymerase activity assays, single- or double-stranded exonuclease activity assays, or single- or double-stranded endonuclease activity assays.

15. The method of claim 8, wherein said detergent(s) selectively activate DNA polymerase activity.

16-21. (canceled)


SEQUENCE LISTING (12 sequences)

SEQ ID NO: 1 - protein, 832 aa, Thermus aquaticus [sequence omitted]

SEQ ID NO: 2 - protein, 834 aa, Thermus thermophilus
MEAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRRDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGMGEDWLSAKG

SEQ ID NO: 3 - protein, 830 aa, Thermus oshimai
MLPLFEPKGRVLLVDGHHLAYRTFFALKGLTTSRGEPVQAVYGFAKSLLKALKEDGEVAIVVFDAKAPSFRHEAYEAYKAGRAPTPEDFPRQLALIKELVDLLGLVRLEVPGFEADDVLATLAKKAEREGYEVRILSADRDLYQLLSDRIHLLHPEGEVLTPGWLQERYGLSPERWVEYRALVGDPSDNLPGVPGIGEKTALKLLKEWGSLEAILKNLDQVKPERVREAIRNNLDKLQMSLELSRLRTDLPLEVDFAKRREPDWEGLKAFLERLEFGSLLHEFGLLEAPKEAEEAPWPPPGGAFLGFLLSRPEPMWAELLALAGAKEGRVHREADPVGALKDLKEIRGLLAKDLSVLALREGREIPPGDDPMLLAYLLDPGNTNPEGVARRYGGEWKEDAAARALLSERLWQALYPRVAEEERLLWLYREVERPLAQVLAHMEATGVRLDVPYLEALSQEVAFELERLEAEVHRLAGHPFNLNSRDQLERVLFDELGLPPIGKTEKTGKRSTSAAVLELLREAHPIVGRILEYRELMKLKSTYIDPLPRLVHPKTGRLHTRFNQTATATGRLSSSDPNLQNIPVRTPLGQRIRKAFIAEEGHLLVALDYSQIELRVLAHLSGDENLIRVFREGKDIHTETAAWMFGVPPEGVDGAMRRAAKTVNFGVLYGMSAHRLSQELSIPYEEAAAFIERYFQSFPKVRAWIAKTLEEGRKKGYVETLFGRRRYVPDLNARVKSVREAAERMAFNMPVQGTAADLMKLAMVKLFPRLRPLGVRILLQVHDELVLEAPKARAEEAAQLAKETMEGVYPLSVPLEVEVGMGEDWLSAKA

SEQ ID NO: 4 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 5 - protein, 876 aa, Bacillus stearothermophilus
MKNKLVLIDGNSVAYRAFFALPLLHNDKGIHTNAVYGFTMMLNKILAEEQPTHILVAFDAGKTTFRHETFQDYKGGRQQTPPELSEQFPLLRELLKAYRIPAYELDHYEADDIIGTMAARAEREGFAVKVISGDRDLTQLASPQVTVEITKKGITDIESYTPETVVEKYGLTPEQIVDLKGLMGDKSDNIPGVPGIGEKTAVKLLKQFGTVENVLASIDEIKGEKLKENLRQYRDLALLSKQLAAICRDAPVELTLDDIVYKGEDREKVVALFQELGFQSFLDKMAVQTDEGEKPLAGMDFAIADSVTDEMLADKAALVVEVVGDNYHHAPIVGIALANERGRFFLRPETALADPKFLAWLGDETKKKTMFDSKRAAVALKWKGIELRGVVFDLLLAAYLLDPAQAAGDVAAVAKMHQYEAVRSDEAVYGKGAKRTVPDEPTLAEHLVRKAAAIWALEEPLMDELRRNEQDRLLTELEQPLAGILANMEFTGVKVDTKRLEQMGAELTEQLQAVERRIYELAGQEFNINSPKQLGTVLFDKLQLPVLKKTKTGYSTSADVLEKLAPHHEIVEHILHYRQLGKLQSTYIEGLLKVVHPVTGKVHTMFNQALTQTGRLSSVEPNLQNIPIRLEEGRKIRQAFVPSEPDWLIFAADYSQIELRVLAHIAEDDNLIEAFRRGLDIHTKTAMDIFHVSEEDVTANMRRQAKAVNFGIVYGISDYGLAQNLNITRKEAAEFIERYFASFPGVKQYMDNIVQEAKQKGYVTTLLHRRRYLPDITSRNFNVRSFAERTAMNTPIQGSAADIIKKAMIDLSVRLREERLQARLLLQVHDELILEAPKEEIERLCRLVPEVMEQAVTLRVPLKVDYHYGPTWYDAK

SEQ ID NO: 6 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 7 - protein, 774 aa, Pyrococcus Kodakaraensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 8 - protein, 779 aa, N Thermococcus barossii
MILDVDYITEDGKPVIRVFKKDKGEFKIEYDREFEPYIYALLRDDSAIEEIEKITAERHGKVVKVKRAEKVKKKFLGRSVEVWVLYFTHPQDVPAIRPDKIRKHPAVIDIYEYDIPFAKRYLIDKGLIPMEGDEELKLMSFDIETLYHEGEEFGTGPILMISYADESEARVITWKKIDLPYVDVVSTEKEMIKRFLKVVKEKDPDVLITYDGDNFDFAYLKKRCEKLGVSFTLGRDGSEPKIQRMGDRFAVEVKGRIHFDLYPAIRRTINLPTYTLEAVYEAVFGKPKEKVYAEEIATAWETGEGLEGVARYSMEDARVTYELGREFFPMEAQLSRLIGQGLWDVSRSSTGNLVEWFLLRKAYERNELAPNKPDERELARRRGGYAGGYVKEPERGLWDNIVYLDFRSLYPSIIITHNVSPDTLNREGCKSYDVAPQVGHKFCKDFPGFIPSLLGNLLEERQKIKRKMKATLDPLERKLLDYRQRAIKILANSFYGYYGYARARWYCKECAESVTAWGREYIEMVIRELEEKFGFKDLYADTDGLHATIPGADRETVKKKDLEFLNYINPKLPGLLELEYEGFYSRGFFVTKKKYAVIDEEGKITTRGLEIVRRDWSEIAKETLARVLEAILRHGDVEEAVRIVKEETEKLSKYEVPPEKLVITEQITRELKDYKATGPHVAIAKRLAARGIKIRPGTVISYIVLKGSGRIGDRAIPFDEFDPTKHRYDADYYIENQVLPAVERILRAFGYKKEDERYQKTRQVGLGAWLGMGGERLKL

SEQ ID NO: 9 - protein, 779 aa, Thermococcus barossii
MILDVDYITEDGKPVIRVFKKDKGEFKIEYDREFEPYIYALLRDDSAIEEIEKITAERHGKVVKVKRAEKVKKKFLGRSVEVWVLYFTHPQDVPAIRPDKIRKHPAVIDIYEYDIPFAKRYLIDKGLIPMEGDEELKLMSFDIETLYHEGEEFGTGPILMISYADESEARVITWKKIDLPYVDVVSTEKEMIKRFLKVVKEKDPDVLITYDGDNFDFAYLKKRCEKLGVSFTLGRDGSEPKIQRMGDRFAVEVKGRIHFDLYPAIRRTINLPTYTLEAVYEAVFGKPKEKVYAEEIATAWETGEGLEGVARYSMEDARVTYELGREFFPMEAQLSRLIGQGLWDVSRSSTGNLVEWFLLRKAYERNELAPNKPDERELARRRGGYAGGYVKEPERGLWDNIVYLDFRSLYPSIIITHNVSPDTLNREGCKSYDVAPQVGHKFCKDFPGFIPSLLGNLLEERQKIKRKMKATLDPLERKLLDRYQRAIKILANSFYGYYGYARARWYCKECAESVTAWGREYIEMVIRELEEKFGFKDLYADTDGLHATIPGADRETVKKKDLEFLNYINPKLPGLLELEYEGFYSRGFFVTKKKYAVIDEEGKITTRGLEIVRRDWSEIAKETLARVLEAILRHGDVEEAVRIVKEETEKLSKYEVPPEKLVITEQITRELKDYKATGPHVAIAKRLAARGIKIRPGTVISYIVLKGSGRIGDRAIPFDEFDPTKHYDRADYYIENQVLPAVERILRAFGYKKEDERYQKTRQVGLGAWLGMGGERLKL

SEQ ID NO: 10 - protein, 561 aa, Thermus aquaticus
MLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 11 - protein, 561 aa, Thermus thermophilus
MLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRRDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNYGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGMGEDWLSAKG

SEQ ID NO: 12 - protein, 509 aa, Thermus aquaticus
MLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQWLAIPYEEAQAFIERYFQSFPKVRAWIEKTLEEGRRRGYVETLFGRRRYVPDLEARVKSVREAAERMAFNMPVQGTAADLMKLAMVKLFPRLEEMGARML